(19) United States
(12) Patent Application Publication
(10) Pub. No.: US 2007/0031509 A1
(43) Pub. Date: Feb. 8, 2007

(54) Breakthrough to cure psoriasis, psoriatic arthritis and treatment of other unrelated skin disorders, and external rectal and genital itching

(76) Inventor: Laxman S. Sundae, Rosemount, MN (US)

Correspondence Address: LAXMAN S. SUNDAE, 2055-128th St. W, Rosemount, MN, 55068, US

(21) Appl. No.: 11/136,198

(22) Filed: Aug. 4, 2005

(51) Int. Cl. A61K 33/34; A61K 33/32; A61K 36/886

(52) U.S. Cl. 424/638; 424/642; 424/744

(57) ABSTRACT

This invention provides a novel method of treating psoriasis in humans by topical administration of a mixture consisting of copper lactate [Cu (C₃, H₅ O₃)₂. 2H₂O], zinc hydroxide Zn(OH)₂, trace amounts of cupric oxide and purified water by blunting the action of T cells present in the bleeding points located in and around the psoriatic lesions. When the bleeding points are absent in psoriatic lesions, this composition eliminates primary psoriatic symptoms such as inflammation, itching, rete ridges and tough red skin patches without curing psoriasis. The size and population of psoriatic lesions is also reduced. This composition is also very effective for providing instant relief from skin rash and itching.

### BACKGROUND OF INVENTION

1. Nature of Investigation

This invention is directed to curing and/or treatment of psoriasis or skin rash or itching.

2. Background Art

Psoriasis is a chronic (life long, recurring) Stone Age disease. It causes one or more raised, localized red plaques often topped by silvery scales and a distinct border between the red patches and normal skin. The National Institute of Arthritis and Musculoskeletal and Skin Disease (NIAMSD) and the National Psoriasis Foundation (NPF) have estimated that 2-2.5 percent of world population carries this disease. In the U.S., roughly 5.8 to 7.5 million people are afflicted with this disease, making it one of the most common skin disorders.

Psoriasis is a symptom of a white blood cell (T cell) disorder manifested in the form of a skin disease, i.e., the underlying cause of psoriasis is inflammation of the T cells (1). Researchers have concluded that T cells go berserk and move into the dermis and epidermis layers in large numbers, punching little holes through the membrane that separate the two skin layers. The lowest layer (basal layer) where the skin cells are born is damaged, and this switches on the whole hyper-proliferating process, increasing skin turnover rate from 28 days to 3 or 4 days (1).

Baker presents commonly accepted theory that psoriasis is a disease of abnormal keratinocyte proliferation induced by T cells (2). Recently, Bowcock et. al. using data from a NPF tissue bank identified three of the first genes associated with psoriasis(3).

In the past decade, I had noticed a quick turnover rate and resulting change in the symptoms: diseased skin became impervious with white encrusted lesions or thick and tough silvery patches of dead skin cells atop the thickened red skin or plaque. Over the past 16 years, I have noticed that the patches of silvery scales usually thicken and flake off. But the forced removal of silver scales either left behind the fine bleeding points or red thickened skin. Itching, inflammation, and pain under and around the white encrusted lesions and bleeding points were routine part of my life with psoriasis.

Despite their common origin, psoriatic symptoms such as scaling, itching, red skin, bleeding points, and pustules (noninfectious white blood cells) require separate treatment methods. Nonetheless, with a few exceptions, previous investigators have attempted to find a treatment for itching but have failed to address the remaining symptoms such as the inflammation of skin and bleeding points. As of now, more than 13,194 patents have been obtained without suggesting successful cure psoriasis and its symptoms.

In most cases, the current treatments are too expensive and completely ineffective. Most of the topical application of moisturizing creams, ointments and lotions containing coal tar, lactic acid, urea, anthralin, salicylic acid, derivatives of vitamins A and D, and steroids do not penetrate silvery patches or dislodge the tough plaques. All of the potions used to remove the dead cells are buffered by or contain lubricating agents. They require too many applications and are somewhat ineffective in quickly removing the silvery patches with one or two applications.

Promoters of exorex claim that their product (coal tar and fatty acids emulsion from banana peel) easily penetrate the skin to reach the basal layer, yet, they don't recommend its application to open wounds or the bleeding points (4). This is an implausible paradox, because for any product to treat psoriasis, it has to come in contact with inflamed T cells. Regardless of their laudable claims, the coal and other mineral impurities found in coal tar are unable to penetrate the skin layers. Otherwise, thousands and thousands of coal miners and workers loading and unloading, handling coal and coal tar for living, including this investigator would be presenting with the submicroscopic coal particles and minerals impurities in their blood streams.

After months of gentle massaging, over-the-counter drugs (OTCs) are successful in peeling off or removing the silvery patches from the top of reddened skin. Exposure of fresh reddened skin to ambient temperature temporarily eliminates itching.

In the past, a few researchers have attributed copper and zinc deficiencies in skin as the cause of psoriasis. These elements are essential to enhance the skin's healing process and they display some anti-inflammatory characteristics (5-7). Several patents suggest use of cupric oxide, yet no product containing copper and zinc compounds has been successful in treating psoriasis.

Glassman et. al., U.S. Pat. No. 6,827,943 suggest use of a compositions containing 21% to about 40% urea and trace amounts of zinc, copper and manganese in the form of their respective pharmaceutically acceptable salts selected from groups consisting of acetates, bromides, carbonates, chlorides, citrates, gluconates, glycinates, glycerophospates, lactates, iodides, nitrates, sulfates, and mixture thereof.

This patents lists 36 chemicals having vary different chemical and physical properties. With such unusually high number of variables ranging from 1 ppm to 0.5 wt.-% and with combinations and permutations of these many variables it is possible to manufacture more than 500,000 individual products. They fail to identify a specific formula that can be used to treat psoriasis. Thusly, this patent is lacking full disclosure and is so vague and overbroad that even an expert engaged in the field of prior art would not be able to decipher a composition or formulate a product to treat or cure immunological skin disorders. Furthermore, trace amounts of copper and zinc salts are shrouded by large amount of urea and inactive ingredients in this composition, making it impossible for one of the imaginary candidate to penetrate psoriatic lesions to reach inflamed T cells. Furthermore, this composition contains trace amounts of zinc, copper and manganese in the form of their respective pharmaceutically acceptable salts, it should be noted that such low doses are too low to be effective.

Furthermore, it should be pointed out that manganese, copper and zinc salts in trace amounts suggested by Glassman et. al. are part and parcel of all vitamin supplements and do not treat or cure psoriasis or any other related skin disorders. U.S. Pat. No. 6,630,158 also describes usefulness of essential elements for improving or maintaining healthy skin.

Peshoff in U.S. Pat. No. 6,660,306, based on his review of patents and scientific literature of prior art states that zinc oxide has shown to be an essential catalyst in many biological reactions. He also states that application of zinc oxide to wounds not only corrects local deficiency but also act pharmacologically. This is all-true for wound healing, but has nothing to do with cure or treatment of immunological disorders such as psoriasis.

Based on the shear number of U.S. Pat. No. (13,194) and literature survey, one can safely conclude that treatments for primary symptoms of psoriasis are many and varied, but no claim has been made to cure this disease.

Bowel incontinence is an involuntary or accidental leakage of liquid stool from the bowel. It occurs in people of all ages. It is more common than previously thought and it causes serious rectal itching. The current treatments are too expensive and slow to treat itching.

### Objective

Accordingly, the first object of this invention was to find a cure for different types of psoriasis, which can be topically administered by the patients themselves, a composition which can correct local deficiency of copper (Cu) and Zinc (Zn), which can act pharmacologically to either kill over-activated T cells or disrupt communication from T Cells to Cytokine to epidermal cells to prevent hyperproliferation of Keratinocyte.

Second objective was to the determine the suitability of this composition for treatment of eczema and other related skin disorders and provide a way to manage psoriasis and other skin disorders that will cost appreciably much less. The end users, i.e., patients will appreciate the low cost and ease of topical application of this composition.

Further objects will become apparent from the ensuing description and claims.

### UNIQUNESS OF INVENTION AND ADVANTAGES

Patent examination and literature review of prior art show that this invention is very unique and non-obvious for several reasons. Review of prior art shows that there are several OTC's for treating primary symptoms of different types of psoriasis on a very short term basis. However, OTC's are not designed to address the underlying T cells or TNF-Alfa disorders.

It is common knowledge that in 80% of the cases the biologic agents are quite effective in successfully treating symptoms of psoriasis. Nonetheless, when treatment is stopped, psoriasis erupts quite rigorously, far worst than before the start of treatment requiring repeated cycles of treatments. These drugs disable protective aspect of the immune system, leaving patients vulnerable to infection and cancer (8).

Inventor herein is using a novel chemical composition consisting of pharmaceutically acceptable salts, an entirely different scientific approach to prevent the hyperproliferation of epidermal cells by correcting local deficiency of Cu and Zn. This composition and methods of correcting local deficiency of Cu and Zn are unknown to previous patent holders and scientists working in this field.

In comparison to biologic agents, systematic medicines and the OTC's, the test composition provides a very inexpensive treatment of psoriasis, psoriatic arthritis, skin rash itching without any bad side effects or rebound. Since, there is no danger of cancer; there is no need of frequent testing for cancer or any other disease.

Theoretical approach undertaken in this innovation is directed toward attaching copper lactate, zinc hydroxide, cupric oxide and zinc oxide composition to T cells and epidermal cells, two major parts of immune system.

The final product of copper, zinc and yogurt used in manufacturing this medicine are inexpensive and will drastically lower the cost of curing and/or treatment of different types of psoriasis and other skin disorders.

### SUMMARY

A fair amount of composition containing 60% copper lactate [Cu (C₃ H₅ O₃)₂. 2H₂O], trace amounts of cupric oxide (Cu₂O), 20% to almost 30% zinc hydroxide Zn(OH)₂ and 10% purified water was gently massaged 3 to 4 times a day on to the bleeding points located on the symmetrical silvery patches on my elbows. Test results showed that the psoriasis was completely and totally cured within 10 days. Since then, nearly three years have gone by and the psoriasis has not returned to the previously infected areas and the skin is smooth and healthy. Please see reference points L₁ and L₂ in FIG. 2.

During the testing phase, six lesions developed outside the outer parameters of lesions L₁ and L₂ without producing bleeding points. In each of these cases, scratching off the tops of silvery patches created artificial bleeding points. Test composition was applied to artificially created bleeding points and soon after the psoriatic patches disappeared.

Even though the theoretical mechanism is not understood, in all probabilities the copper lactate [Cu (C₃, H₅ O₃)₂. 2H₂O], and zinc hydroxide Zn(OH)₂, when topically applied to bleeding points, blunted the action of over activated T cells and terminated the chain of communication between the T Cells, Cytokines and Keratinocyte by correcting local deficiency of Cu and Zn.

Additional tests show that this invention provides a unique medication for the treatment of psoriatic arthritis, skin rash and itching.

This innovation also provides the quickest means to eliminate itching of external rectal (caused by bowl leakage) and genital parts within less than 45 seconds of a single topical application, making it the most efficient and economical treatment. Thus, it has a very significant advantage over existing treatments.

Comparative analysis with all OTCs and prior art shows that this is the only innovation that successfully cures psoriasis by destroying T cells or breaking the link to Cytokines, the major parts of immune system.

### DESCRIPTION OF FIGURES

FIG. 1. Deepak or brass container used to manufacture test composition.

FIG. 2. (a). Lesions L₁ and L₂ were cured during the first phase of study in the last week of May 2004.

(b). Lesions L₃,-L₄ were cured by application of test composition to bleeding points accidentally created by scratching of silvery patches during sleep in the second phase of testing in the summer of 2005.

(C) Lesion L₅ was cured by application of test composition to newly created bleeding point by intentional fingernail scratching of this lesion during the summer of 2005.

FIG. 3. Toward the end of second phase of testing lesions L₆ thru L₈ developed on the outside of outer parameters of previously cured lesions and over a period of four months slowly grew bigger without producing bleeding points.

FIG. 4. Shows an emery board being used to create artificial bleeding points on lesions L₆ thru L₈.

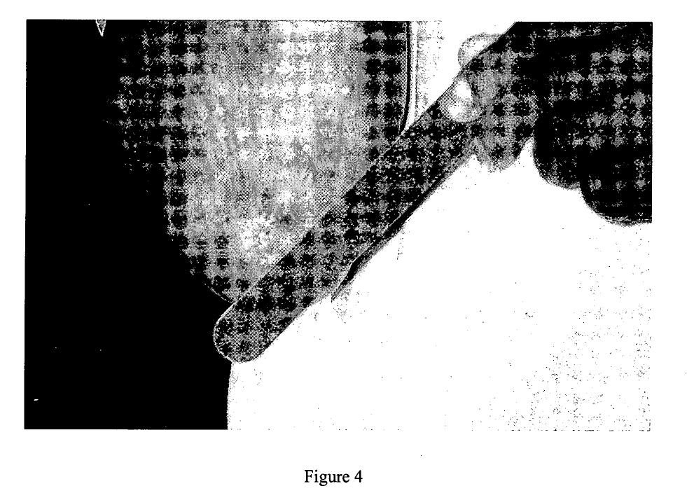

FIG. 5. Lesions L₆ thru L₈ were cured by several application of test composition to artificially created bleeding points during the final phase of testing in the month of September 2005.

Till this date, the psoriasis has not returned to lesions L₁-L₈.

### DETAILED DESCRIPTION OF THE INVENTION

Since 1990, I have had a mild case of symmetrical plaque psoriasis on both of my elbows. Initially, it started as a single cell on my left elbow. A few months later, in the same fashion, another silvery patch developed on my right elbow. Both of these inflammations grew over an area of about 1.5 square-inches. Psoriasis on my right arm is a mirror image of my left elbow.

In the past, to minimize itching, I have tried many types of OTCs in the form of creams, lotions, moisturizers, and ointments without any success. After several years of OTC use, I was fed up with the failures of these regimens and from 2001-2003, to alleviate itching; I hydrated the thickened silvery scales by using warm water and exfoliated the dead cells with a washcloth. Clue for the treatment: During the winter of 2004, I was traveling in northwestern India and did not wish to use untreated water to contaminate the several bleeding points on my elbows. On the last day of my visit, my elbows were itching profusely and I was scratching them constantly to alleviate itching. I told my family that once in a great while I had used “udder cream” to alleviate the itch. One of my family members suggested that for now instead of udder cream I could just use yogurt.

When I was growing up in the Thar Desert in northwest India, my mother had once applied farm made yogurt to a minor burn to stop pain. Fifty years later, I still remember that cool and soothing feeling of that yogurt application; therefore, despite my fear of contamination I decided to give it a try.

I rubbed-in some fresh farm made yogurt with my fingertips on lesions and took some with me to apply later on during the day. The yogurt was accidentally left overnight in a “Lota” (a small brass container). The lactic acid in the yogurt reacted with the Cu and Zn in the Lota and the yogurt got ruined. Nonetheless, just to alleviate the persistent itch and pain, I still decided to use it. Topical applications over the “bleeding points” caused a minor burning sensation for a minute or so. Lo and behold, I noticed that when the contaminated yogurt came in contact with bleeding points, the itch and pain were gone. In contrast to my past experience with the OTCs, the itching and pain disappeared relatively quickly. Therefore, I was somewhat puzzled with the results. I knew that none of the OTCs contained yogurt, therefore, the uniqueness of the result intrigued me and gave me reason to verify the results again.

### Experimental Procedure

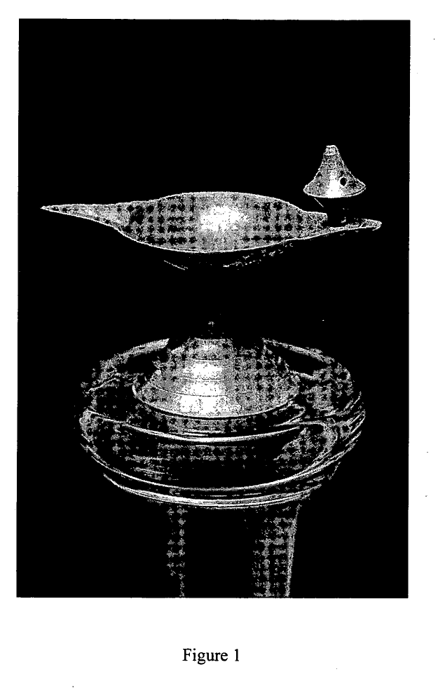

Manufacturing of copper-zinc composition: The yogurt used to prepare this composition was non-fat yogurt available in any grocery store and the brass container (Deepak) shown in FIG. 1, was purchased from a Goodwill Industries store. The “Deepak” shown in FIG. 1 is like any other such container commonly used in many Indian homes to worship Gods. It is made of 78% Cu, 21% Zn and 1% Al by weight.

It is essential to use containers made from coarse and unpolished alloy to expedite chemical reaction. The final product for topical applications at minimum should contain 60 to 70% copper lactate, 20% to almost 30% zinc hydroxide and 10% inactive ingredients such as purified water and Aloe Vera.

The research composition used in this test series was prepared by placing a small amount of fresh yogurt having a pH of 4.1-4.2 and containing live active cultures, including Acidophilus and bifidus into a brass container for 18-24 hours at room temperature. During this period lactic acid (C₃H₆O₃) in yogurt reacted with Cu and Zn and the yogurt near the contact surface became bluish-green, indicating that a chemical reaction has taken place producing copper lactate {[Cu (C₃, H₅ O₃)₂. 2H₂O]}, and zinc hydroxide {(Zn(OH)₂}. Test composition has somewhat unpleasant metallic taste.

The manufacturing process of these compounds or mixtures is given only as an example. Many other upscale processes are possible to increase the production. Most of the base chemicals like cupric oxide, zinc hydroxide and zinc oxide are readily available from standard chemical catalogs.

### Treatment of Psoriasis

I had worked as a research engineer for the U. S. Department of Interior for 30 years. Based on my background, I was intrigued by the anomalous and quick reduction in itching and inflammation by a previously unknown composition made from yogurt. Therefore, I became interested in reexamining my findings. A few months after returning from India, I decided to determine the feasibility of using yogurt and/or copper-zinc composition to alleviate itching. The procedure to prepare test composition is briefly described in the subsection titled as manufacturing of copper-zinc composition above.

I am a 64-year-old male. I have had plaque psoriasis, psoriatic arthritis, and diabetes for more than 16 years and coronary heart disease for more than 5 years. All times material to this investigation I was on Humalog and Lantus Insulin, Vitamin B12, Metformin, Amryl, Plavix, Aspirin, lmdur, Atenolol, Zocor and very strict heart healthy and diabetic diets. All times material to this patent application, I have taken Omega-3 fish oil 1000 mg, copper 2 mg, manganese 2 mg and zinc 15 mg found in multivitamin/multimineral supplement for adults since September 2000.

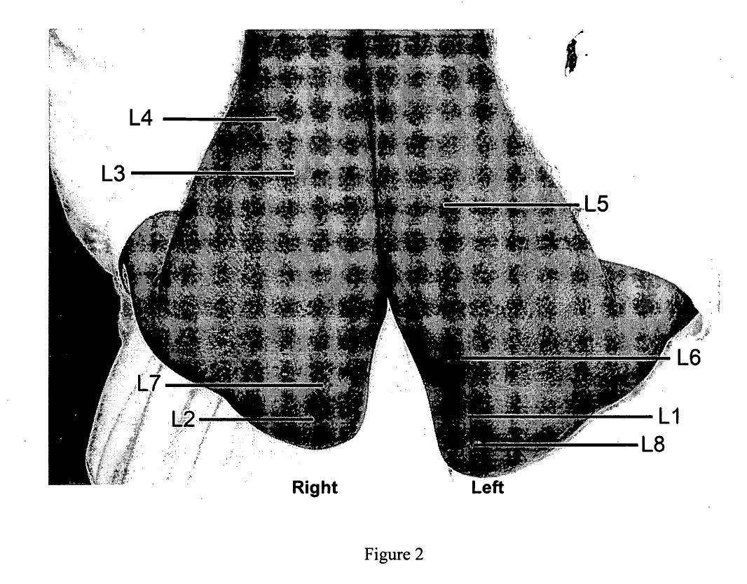

Test results: Within 48 hours of topical applications, I noticed that all silvery scales fell off, dome-like bubble formed over the bleeding points, and the skin underneath the silvery lesions became red, swollen and severally inflamed. But these symptoms disappeared within four days of first application. With 3 or 4 additional daily applications over the next 5 days, psoriatic lesions disappeared completely leaving behind two darker spots. Please see areas marked as L₁ and L₂ in FIG. 2.

During the testing period, three isolated silvery patches developed that were approximately 2-5 millimeter in diameter. See L₃, L₄ and L₅ in FIG. 2. Due to accidental scratching during sleep, I created bleeding points on lesions L₃ and L₄. When test composition was applied to these bleeding points, the psoriasis disappeared leaving behind two dark spots. See L₃₋₄ in photographs FIG. 2. This was somewhat encouraging, therefore, I scratched off lesions L₅ and created a bleeding point on it. Test composition was applied and this lesion also disappeared permanently.

Toward the later stages of testing, I noticed that the lactic acid in yogurt had corroded and thinned the interior surface of brass container where it had come in contact with the metallic surface of container. Further usage made a hole through its side rendering test container useless for further research.

At this stage, it was believed that in all probability the copper lactate [Cu (C₃, H₅ O₃)₂. 2H₂O], and zinc hydroxide Zn(OH)₂ took part either in correcting Cu and Zinc deficiency or by curtailing the activity of over activated T cells. Due to the unorthodox nature of manufacturing process and topical applications, it is impossible to determine the amount of copper and zinc entering the bleeding points.

### Treatment of Psoriatic Arthritis

Just like the mild case of psoriasis, I also had a psoriatic arthritis in my right hand ring finger. As soon as the psoriasis symptoms disappeared, so did the mild pain and the swelling of finger caused by psoriatic arthritis.

Similarly, with the recurrence of psoriatic lesions L₃-L₅, the psoriatic arthritis reappeared but in absence of bleeding point, it did not responded to applications of copper lactates. But there is significant difference between two events. Psoriasis did not retrun to same location, while the psoriatic arthiritis reappeared on the same joint.

Surprisingly, during the final phase of this study in the month of September 2005, the psoriatic arthritis symptoms again disappeared with the disappearance of psoriatic lesions. Since than more than several months have gone by without swelling or pain in my right hand ring finger.

### Instant Relief from Ithing Caused by Skin Rash

During the winter of 2004-05, due to profuse sweating, I developed a rash in the skin folds between the genital area and interior portion of thigh. Test composition was topically applied to the affected area and the itching disappeared in less than one minute. Topical application was quite effective and only a couple of treatments were needed to cure the inflammation. Much to my surprise, a few weeks later, similar itching was noticed around the previous locations and the mixture was reapplied and inflammation disappeared with two applications. In each of these cases the top layer of epidermis also peeled off.

### Instant Relief from Rectal Itching

Rectal itching in the instant case was caused by a small amount of fluid leakage from the bowel caused by “low-level-constantly-nagging-mental stress”. As soon as the test composition having 60 to 70% copper lactate and 20 zinc hydroxide and 10% water was applied to the areas affected by fluid leakage, the itching disappeared in less than 45 seconds.

The chemistry and biology of this relief are entirely different from killing of T cells or alteration of skin cells. Duration of cure suggests that (within 45 seconds of topical application) a chemical reaction took place causing a quick change in the pH of fluid leaked into the external areas of rectum from the bowel. This product also works effective to curtail itching caused by uncontrolled type-2 diabetes under the foreskin. When 70% copper lactate, 20% zinc hydroxide and 10% talc powder are applied to prevent itching, it takes more than 5 minutes to eliminate itching.

### No Treatment Possible Without Contact with Beeding Points

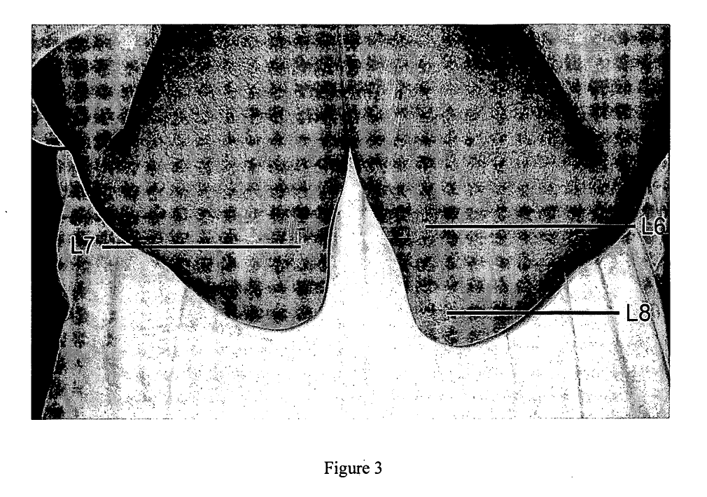

After nine months, during the month of March 2005, psoriasis reappeared one dead cell at a time on the outer parameters of old sites and spread away from the old sites on both hands. Please see reference points L₇ to L₈ in FIG. 3. In fact, over the course of two months, these symmetrical patches have reduced to the size of a U.S. Penny. It is also surprising that psoriasis did not come back on the previously treated lesions L₁ and L₂.

Immediately, I started treating the new growth with the copper lactate [Cu (C₃, H₅ O₃)₂. 2H₂O], and zinc hydroxide Zn(OH)₂ composition. This process has totally inhibited formation of bleeding points. Patches of red (inflamed) skin have decreased in size, there is a significant decrease in thickness of epidermis. But the red skin is tougher and imperforate with very distinct boundaries. Repeated applications have reduced inflammation. Because of a decrease in size of the lesions, the quantity of skin cells being reproduced is lessened without any noticeable decrease in turnover rate. In absence of thick silvery patches, there is no cracking, itching or bleeding, yet, the psoriasis has not disappeared.

Because of lack of test composition's contacts with T cells, psoriasis has not been cured, but hyperproliferation of Keratinocyte has been somewhat reduced.

After ten days of somewhat disappointing results with L₆, L₇ and L₈, this test series was discontinued and it was decided to compare the performance of test composition against the most commonly used OTCs, copper lactate [Cu (C₃, H₅ O₃)₂. 2H₂O], zinc hydroxide Zn(OH)₂. Later portions of these tests were performed to find out whether zinc hydroxide or copper lactate or combination thereof is more effective in attacking and killing of T cells.

### Comparison of Performance of OTCs, Zinc Oxide and Copper Lactate

As mentioned above, the presence and growth of L₆, L₇ and L₈ lesions gave me an opportunity to compare the performance of my invention against the readily available OTCs. As mentioned earlier, coal tar, sea salts and several combinations of acids are used for treatment of psoriasis and eczema.

From June 1966 to March 1996, The U. S. Department of Interior (DOI) employed me as a research engineer. During the course of my employment with DOI, I had conducted research in more than 100 coal and salt mines. I have a sound knowledge of the properties of coal and salts. The following comments are based on my experimentations and my in-depth knowledge of the physical and chemical properties of these minerals. Furthermore, based on my own knowledge and past experience with the OTCs, I became suspicious of the validity of the claims made by the manufactures of OTCs containing coal tar and sea salts. Therefore, I designed a simple experimental procedure to compare effectiveness of readily available OTCs to treat psoriasis. Experimental procedure: Before each topical application, each of the three silvery patches was washed with warm soapy water and towel dried. Each OTC product was gently massaged onto all three silvery patches three times daily. Topical applications were made in accordance with the drug manufacturer's recommendations. At the end of four hours, physical observations were made. Silvery patches were again washed and dried and medication was applied again. Each product was tested for a period of four days. After completion of one test series and start of another, silvery patches were left alone for a period of four days to avoid carryover affects from one product to another.

Immune suppressing medications such as Elidel and Protopic and all other prescription drugs known to potentially contribute to cancer were excluded from this test series. Prescription phototherapy and systemics having different course of action and generally, they are not recommended for mild cases of psoriasis. Therefore, these medications were beyond the scope of this study.

(1). Coal tar compositions: Coal itself is an inert material. It does not chemically or biologically react with the skin, but it has a unique physical property of forming a very strong bond with human skin and other objects. Coal tars found in OTCs strongly adhere to the skin. It requires strong rubbing action or frictional force to remove coal tar from the silvery patches and surrounding skin. Removal of dead skin flakes alleviates itching. To further complicate the situation, in low quantities used herein, coal tar has a very light brown streak. When applied to thinned out silvery patches, in the vast majority of cases the coal tar streak matches perfectly the color of brown or sun tanned skin. This makeover gives a false appearance of treatment.

Therefore, it is not the coal tar, but the frictional forces that assist in the removal of silvery patches and elimination of itching. The hosts of inactive ingredients found in the coal tar products also cause moisturizing and softening of skin giving and the unintended color deception of coal tar streak give a false impression of treatment by coal tar OTCs.

(2). Sea Salts: Sea salts are made up of numerous corrosive chemicals and these chemicals easily react with dead cells and assist in shrinkage and loosening of dead cells. Once the cells have been weakened by corrosion, they easily fall off or can be scrubbed off. Again, this eliminates itching. This phenomenon promotes the flaking of dead cells. Sodium chloride (common salt) and magnesium sulfate are the major components of sea salts and are easily absorbed by the human body, but they do not interact with T cells. Though, not mentioned in literature anywhere, but my personal experience shows that it is also possible to obtain the same results with liquid calcium chloride. This chemical is dirt cheap and pumped from a large underground lake located under the city of Detroit, Mich.

(3). Salicylic, Lactic and Other Acids: Today, these acids are active ingredient of many OTCs like topical steroids, anthralin and coal tar. All acids are corrosive to the skin. There is also a danger of salicylic acid poisoning and other side effects. These OTCs have only limited success in removing dead cells due to the corrosive effect of acids. The lubricating, skin softening, and soothing action by the army of inactive ingredients found in OTCs give false appearance of treatment of psoriasis. When the coal tar is added to these products, again it adds a glow of light brown or sun tanned skin to thinned out silvery patches. The manufacturers of these products have never made any claims for cure of psoriasis.

(4). Steroids (Cortisone): They easily penetrate the healthy skin and lesions and, eliminate itching by analgesic reaction. During testing period, even three or four applications of 1% Hydrocortisone cream not only caused the thinning of the skin, but also damaged the follicles and have prevented hair growth in the thinned out portion of skin.

(5). Urea: Urea may be a good product for treatment of wounds, but 16 years ago, this product was found totally ineffective in treatment of dry, flaky, cracked, and bleeding symptoms of eczema caused by high blood-sugar level in type 2 diabetic patient, e.g., this investigator. This experience proved that urea has no value for treating cracks, fissures and flaky skin caused by immunological skin disorders.

(6) Multivitamins/Mineral Supplements: It should be noted that the cupric and zinc oxides are embodied in all sorts of vitamin supplements, but somehow they don't prevent the activation of T cells. Topical applications of these compositions also proved fruitless to treat psoriasis.

Almost all of the manufacturers of OTCs suggest a 30-day treatment regimen. All topical applications require some sort of rubbing action and it is plausible that 30-days of rubbing actions or frictional force provide temporary relief from the primary symptoms such as scaling and itching. All of the OTCs are lubricating agents, while the warm water has no lubricity, it is a good wetting agent and in rubbing actions it acts as a frictional agent. This difference in physical properties of water is very significant for effective removal of silvery plaques by hydration. 7. Zinc Oxide (ZnO): The second objective of this test series was to determine whether zinc hydroxide or copper lactate was responsible for curing the psoriasis.

Research has attributed zinc deficiency to psoriasis (5-7). In the 1960s, scientists studied the role of zinc dietary supplement but found no link to psoriasis. The zinc oxide is an OTC drug and it has been successfully used for treatment of diaper rash, skin abrasion, chafed, chapped, cracked and wind burned skin and lips. Please see U.S. Pat. No. 6,660,306 for effectiveness of zinc oxide for healing of open wounds and correction of the local zinc deficiency.

A thorough literature review and patent search proves that zinc oxide has not been suggested for topical application for the treatment of psoriasis. Since the zinc is an integral part of test composition, this investigation was extended to determine what effect it has in curing psoriasis.

Zinc hydroxide and zinc oxide have similar properties, but the latter has been extensively used to cure diaper rash without any side effects. Therefore, based on prior knowledge, for extension of this study, an ointment containing 40% zinc oxide was selected for testing. During four topical applications daily lasting over two days, the zinc oxide unexpectedly, but effectively cured a bleeding point. For a while, the skin looked just like new without any side effect but later on the existing silvery patch reappeared on this spot.

Additional topical application further proved that zinc oxide is comparable to any other OTC in reducing inflammation of psoriatic lesions. It should be pointed out that even though the inflammation is completely eliminated by zinc oxide applications, the tough and impervious red patches with very distinct boundaries are left intact. In other words zinc oxide could not penetrate the red patches to reach basal layer. Further experimentation with zinc oxide revealed that it did not cure psoriasis.

8. Copper Lactate: Even though during this testing phase, no bleeding points were developed, it was still decided to explore the impact of copper lactate alone on psoriatic lesions. Copper lactate for this test series was produced by reacting yogurt in a copper container rather than a brass utensil.

After the first day of applications, a significant reduction in swelling was noticed. After three days of topical application, silvery scales fell off leaving behind minimum pain. After three days of applications, the thickened red skin started thinning and at this point in time, pain and swelling were completely gone. After five days of application, there were no silvery patches left to create an artificial bleeding point, but daily application continued. After seven days of topical applications, 20% reduction was noticed in psoriatic lesions. Comparison of superiority of copper lactate over zinc oxide:
1. After ten days of daily applications, the silvery patch from one of the psoriatic lesions flaked off.
2. Red thickened skin from this patch has completely disappeared.
3. Remaining two silvery lesions have been reduced in size by 20%.
4. Further topical applications have not eliminated silvery patches on the remaining two lesions.
5. At this stage the silvery patches are so smooth that it is not possible to scratch the skin to produced bleeding points.

This suggests that even though copper lactate is not penetrating damaged skin to reach T cells, somehow it has reacted with epidermal keratinocytes to eliminate the red thickened skin and reduce the size of silvery patches. There is also a very small possibility that copper lactate is working on damaged skin cells and effectively removes red thickened skin by disrupting Keratinocyte hyperproliferation.

### Treatment of Psoriasis by Exposing Bleeding Points

From May 2005 to September 2005, psoriatic lesions L₆, L₇ and L₈ mentioned above were left alone. During this period, they grew in size without producing any bleeding points. Therefore, to get around the absence of bleeding points and to keep up with the low-tech theme, an emery board having course (80 grit) and fine sides (120 grit) was used to file away the silvery scales and to expose plaques. See FIG. 3. This technique exposed fine bleeding points from underneath the plaques. All of the bleeding points were less than 1 mm in diameter and they only became visible because of bleeding.

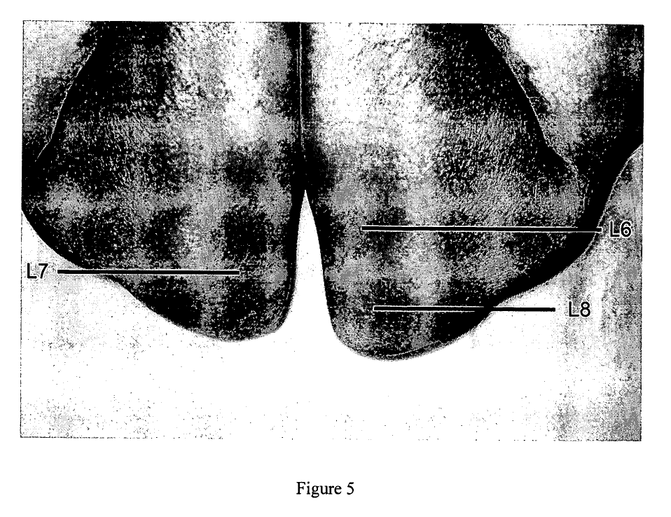

Test composition was quickly applied to cover the bleeding points and surrounding plaques. Even though these bleeding points were very small in size, minor burning sensation was still felt for two or three minutes. Within 5-days of erratic applications, a large portions lesions were cured. A few days later, same procedure was repeated on remaining plaques or lesions. It should be pointed out that because of location of lesions on my elbows, it was not easy to create bleeding points with one or two tries. But with a little patience and little extra filing, a few more bleeding points were created and the test composition was applied till the remaining psoriasis patches cleared up. See FIG. 5. Since than almost a year has gone by and the psoriasis has not reoccurred.

Test history shows that to cure psoriasis, each and every silvery patches, regardless of its size, has to be treated and eliminated, otherwise even a small pinhead will develop into a full blown silvery patch.

Psoriasis occurs all over the body. It is not wise to use topical application on the top of eyelids, inside of mouth, head, soles and palms. In these situations, it may be easier to administer test composition by transdermal, transnasal, intrathecal or subcutaneously or intralesional injection.

### CONCLUSIONS, RAMIFICATIONS AND SCOPE

I realize that psoriasis is a Stone Age disease and the medical community accepts it as a chronic life long disease with well-documented history of remissions and reoccurrences. Nonetheless, my claim of cure for psoriasis is based on solid science and is free from investigator's bias. My conclusion for cure relates to correcting copper and zinc deficiencies in psoriatic lesions. My experimental procedure is well designed.

During each testing phase, I allocated generous lapse of time to avoid experimental bias on my part and rule out possibilities of remission and placebo effects. My findings are well documented with photographic evidence. This investigation was started almost three years ago, testing was conducted over a period of two years and the psoriasis has not returned to any of the lesions. Currently, I am free of psoriasis.

Test composition herein combines the theoretical advantages of two biologic agents Alefacept and Etancercept by blocking over activation of T-Cells and by blocking tumor necrosis factor-alpha and thereby interfering with a key cytokine that contributes to the development of psoriasis. Furthermore, test results show that a series of low-tech treatments lead to cure of psoriasis.

The users will greatly appreciate relief from physical and psychological miseries imposed by psoriasis. From manufacturing point of view, some products are so great that they promote themselves; therefore, advertising costs for test composition will be minimal costs.

The scope of this investigation should be determined not by the examples given, but by the appended claims and their legal equivalents

1-13. (canceled)

14. A method of treating psoriasis in humans comprising topically administering to the bleeding points in the effected area of the skin of human in need of treatment a composition consisting of about 60% copper lactate by weight, about 20% zinc hydroxide by weight, trace amounts of cupric oxide, about 10% purified water by weight and Aloe Vera whereby said administration is effective in treating psoriasis and alleviating one or more symptoms associated with psoriasis.

15. A method of treating psoriatic arthritis in humans comprising topically administering to the psoriatic lesions of the skin of human in need of treatment a composition consisting of about 60% copper lactate by weight, about 20% zinc hydroxide by weight, trace amounts of cupric oxide, about 10% purified water by weight and Aloe Vera where by said administration is effective in alleviating one or more symptoms associated with psoriatic arthritis.

16. A method of treating itching of human skin comprising topically administering to the effected area of the skin of human in need of treatment a composition consisting of 60% copper lactate by weight, 20% zinc hydroxide by weight, trace amounts of cupric oxide, 10% purified water by weight and Aloe Vera where by said administration is effective in alleviating one or more symptoms associated with itching.

17. A method of treating skin rash in humans comprising topically administering to the effected area of the skin of human in need of treatment a composition consisting of 60% copper lactate by weight, 20% zinc hydroxide by weight, trace amounts of cupric oxide, 10% purified water by weight and Aloe Vera where by said administration is effective in alleviating one or more symptoms associated with skin rash.
